(19) United States
(12) Patent Application Publication
(10) Pub. No.: US 2010/0268139 A1
(43) Pub. Date: Oct. 21, 2010

(54) Cervical Collar With Kyphosis Adjustment

(76) Inventor: Geoffrey C. Garth, Long Beach, CA (US)

Correspondence Address: FISH & ASSOCIATES, PC;ROBERT D. FISH, 2603 Main Street, Suite 1000, Irvine, CA, 92614-6232, US

(21) Appl. No.: 12/764,824

(22) Filed: Apr. 21, 2010

Related U.S. Application Data
(60) Provisional application No. 61/171,131, filed on Apr. 21, 2009.

(51) Int. Cl. A61F 5/00

(52) U.S. Cl. 602/18

(57) ABSTRACT

A cervical collar can be adjustable with respect to a wearer's head and neck, and include a back portion positionable posterior to a wearer's neck. The collar can also include a member that extends anteriorly relative to the back portion, and can have a mechanism adapted to reposition a prominence of the member superiorly, inferiorly, anteriorly, and posteriorly relative to the portion while the collar is installed on the wearer. A method of enforcing a lordotic curve in a wearer's cervical spine is also disclosed including installing a cervical collar on the wearer, adjusting the cervical collar to fit the wearer's head and neck, and positioning a member so that the member extends anteriorly with respect to the back portion to an extent that is independent of a tightness of the collar about the neck.

The application claims the benefit of priority to co-pending U.S. provisional application with Ser. No. 61/171,131 filed on Apr. 21, 2009. This and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

### FIELD OF THE INVENTION

The field of the invention is cervical collars.

### BACKGROUND

Cervical collars are medical devices that promote the healing of the spinal cord by restricting the movement of a wearer's head and neck. Cervical collars are useful for patients with cervical injuries such as neck sprains or whiplash, patients with conditions that affect the spinal cord such as arthritis or multiple sclerosis, and patients who have had cervical surgery.

Typical cervical collars must be sized to fit a particular patient before being installed on the patient. In order to stabilize the wearer's head and neck, cervical collars must be made of rigid material. As a result, the wearer's head and neck have a decreased range of motion, which can lead to pain and stiffness in the cervical spine. Such stiffness also increases the wearer's risk of developing cervical kyphosis, a condition in which some or all of the cervical spine's natural lordotic curve is lost.

Several prior art patents and publications describe cervical collars for promoting a natural lordotic curve that are adjustable after installation on the wearer.

U.S. Pat. No. 7,090,653 to Moeller (Aug. 15, 2006) provides a cervical collar with an adjustable tab in the back panel to apply pressure to the wearer's neck. The tab is preferably coupled to a strap that forces the tab anteriorly against the wearer's neck when pulled. Moeller's device, however, is limited in its usefulness because the position of the tab, and hence the extent of the lordotic curve, can only be controlled by the tightness of the strap. The pressure supplied by the tab can only be directed in one direction, which limits the effectiveness of the tab in promoting a natural lordotic curve.

The cervical collars described in U.S. Pat. Nos. 6,896,662 and 6,500,136, and U.S. Pat. Pub. 2006/0135897, offer a slight improvement in that the amount of pressure applied to the neck is independent of the tightness of a strap.

U.S. Pat. No. 6,896,662 to Heffez (May 24, 2005) describes serially attaching one or more pads, one on top of the other, to a posterior force applicator on the back of a neck brace. The more pads attached to the brace, the greater the extent of the lordosis adjustment. U.S. Pat. No. 6,500,136 to Meyer (Dec. 31, 2002) teaches a collar with a cervical sling member engaged to the back of the wearer's neck and adjustable using a pair of control knobs. When the knobs are turned in one direction, the sling member is moved toward the wearer's neck to apply an anteriorly directed force of a prescribed level. U.S. Pat. Pub. 2006/0135897 to Dellanno (Jun. 22, 2006) discloses a collar with an inflatable lordosis correction assembly. The assembly comprises a plurality of separate adjoining pads arranged along the back of the wearer's neck that can be selectively inflated to apply pressure to a particular vertebra.

However, the above devices are limited in that the pressure applied can only be directed anteriorly, rather than superiorly or inferiorly, towards the neck. Consequently, the devices cannot be dynamically positioned in a variety of ways to improve the particular lordotic curve of an individual wearer's neck.

Thus, there remains a considerable need for cervical collars adjustable after installation on a wearer, having a kyphosis adjustment that is independent of the tightness of the collar about the wearer's neck and dynamically positionable in multiple directions with respect to the wearer's neck.

### SUMMARY OF THE INVENTION

The inventive subject matter provides apparatus, systems and methods in which a cervical collar has a kyphosis adjustment that is independent of the tightness of the collar about a wearer's neck, and that can be adjusted superiorly, inferiorly, anteriorly, or posteriorly, while the collar is installed on the wearer. Preferred cervical collars include a back portion that can be positioned posteriorly to a wearer's neck.

Contemplated cervical collars can have a member that that extends anteriorly relative to the back portion to an extent that is independent of a tightness of the collar about the wearer's neck. The collar can further include a mechanism that is adapted to reposition a prominence of the member superiorly, inferiorly, anteriorly, and posteriorly relative to the portion while the collar is installed on the wearer.

Optionally, the cervical collar can include a chest piece that supports at least a portion of the weight of the wearer's head, and a front portion. In some contemplated embodiments, the front portion can be coupled to the back portion using a tightenable strap or other commercially suitable fastener(s). However, it is contemplated that the front portion can be continuous with the back portion, and may or may not have a detachable portion to allow separation of the continuous piece. Such detachable portion could be releasably coupled using one or more commercially suitable fastener(s) including, for example, clips, snaps, hooks and loops, and any combination(s) thereof.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

In one aspect, a method of enforcing a lordotic curve in the cervical spine of a wearer can include the step of installing a cervical collar on the wearer. Once the cervical collar is installed on the wearer, the cervical collar can be adjusted to fit the wearer's head and neck.

A member may be positioned so that the member can extend anteriorly with respect to a back portion of the collar, and to an extent that is independent of the collar's tightness about the wearer's neck. It is contemplated that while the collar is installed on the wearer, a prominence of the member can be repositioned superiorly, inferiorly, anteriorly, and posteriorly relative to the back portion of the collar.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a close-up view of an embodiment of a member.

FIG. 2 is a side view of an embodiment of a cervical collar.

FIG. 3 is a close-up view of an embodiment of a mechanism for repositioning the prominence of the collar's member.

FIG. 4 is a perspective view of an embodiment of a cervical collar.

FIG. 5 is a back view of an embodiment of a cervical collar.

FIG. 6 is a side view of an embodiment of a cervical collar.

FIG. 7 is a close-up view of an alternate embodiment of a member.

FIG. 8 is a flowchart of a method for enforcing a lordotic curve in a wearer's cervical spine.

### DETAILED DESCRIPTION

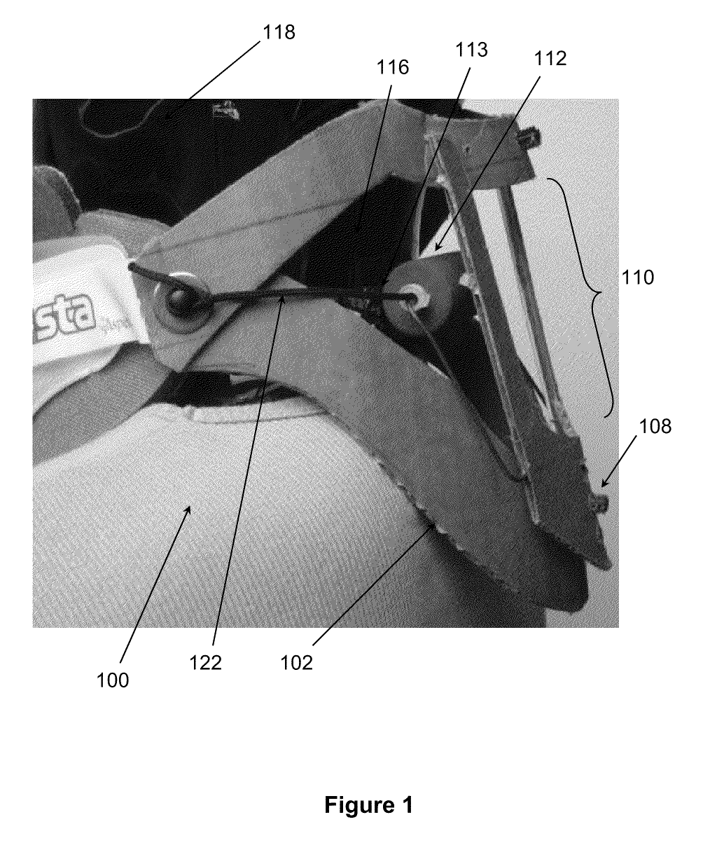

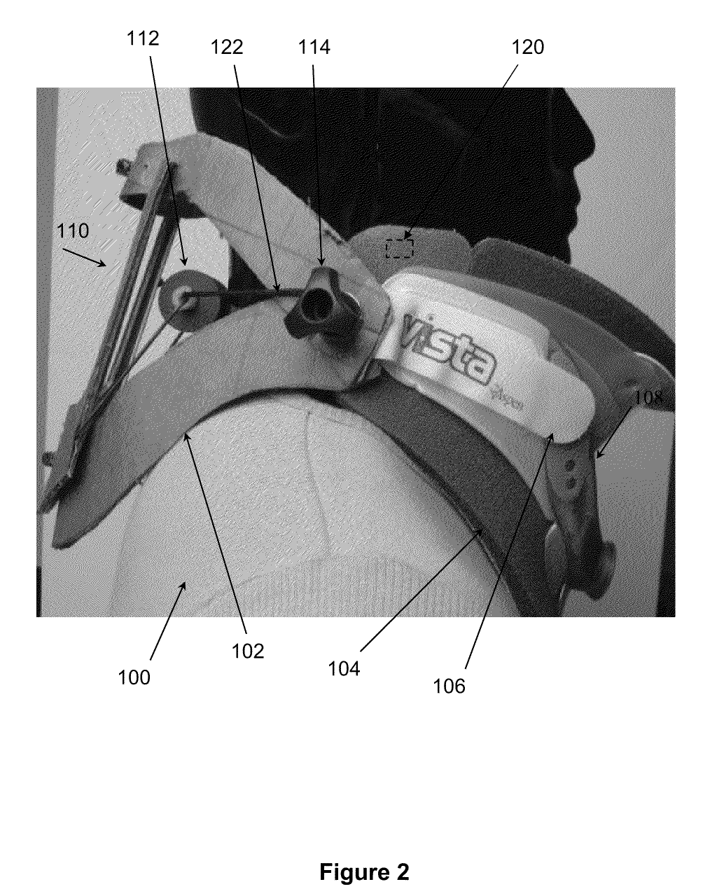

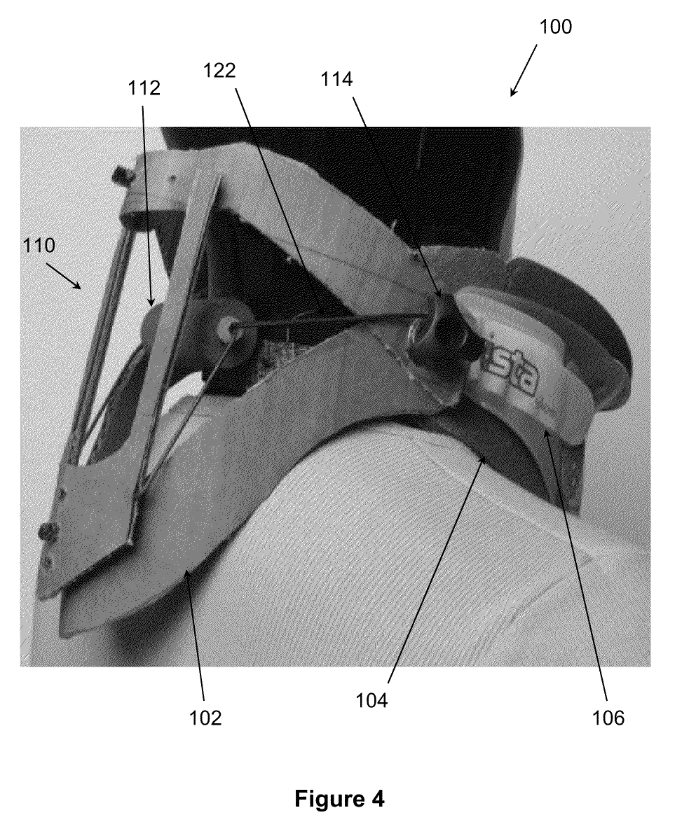

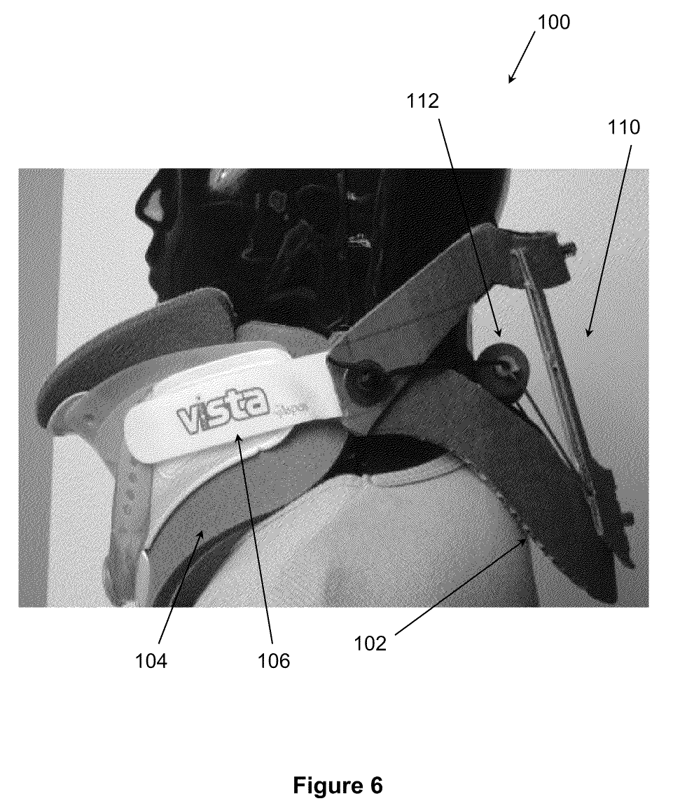

In FIGS. 1-6, a cervical collar 100 is shown that has a back portion 102, which can be positioned posteriorly relative to a wearer's neck 116. Back portion 102 can be permanently affixed to or otherwise coupled to a front portion 104, and the collar 100 can be tightened about the wearer's neck 116 and head 118 using a tightening mechanism 106 that could comprise a strap or other commercially suitable mechanism or combination(s) thereof. Optionally, the tightening mechanism 106 can be configured to releasably couple back portion 102 to front portion 104 such as by using hooks and loops, for example. The front portion 104 of collar 100 can be continuous with the back portion 102 as a single piece encircling the wearer's head 118 and neck 116.

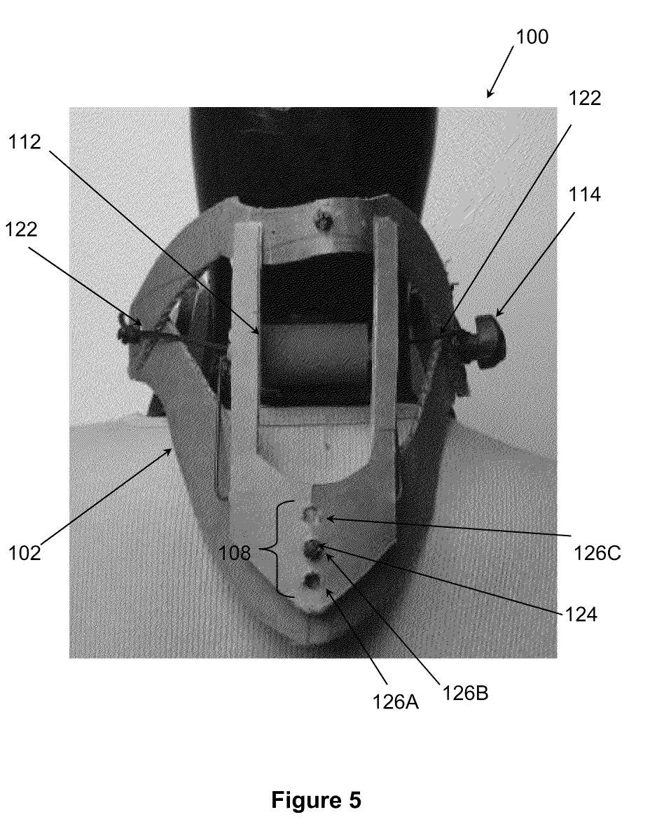

The back portion 102 can include a height adjusting mechanism 108 that allows for adjustment of the height of the back portion 102. As shown best in FIG. 5, the height adjusting mechanism 108 can comprise a pin 124 and a set of slots 126A-126C into which the pin 124 can be inserted. However, the height adjusting mechanism 108 could comprise any commercially suitable mechanism suitable to adjust the height of the back portion 102 including, for example, a dial, a crank, and telescoping pillars with a pin or other locking mechanism.

Optionally, the collar 100 can include a chest piece and/or a back piece that support the wearer's head 118, such as those described in U.S. patent application having Ser. No. 12/469,966 filed on May 21, 2009.

A preferred kyphosis adjustment mechanism 110 comprises a member 112 that extends anteriorly relative to the back portion 102 to an extent that is independent of the tightness of collar 100 about the wearer's neck 116. However, all members suitably shaped for enforcing a lordotic curve in the cervical spine of a wearer are contemplated. It is especially preferred that the member 112 has at least one rounded edge 113 to minimize the wearer's discomfort while the member 112 is pressed against the wearer's neck 116. Further, a member 112 having rounded edge 113 is beneficial for kyphosis adjustment, as the rounded edge 113 mimics the contour of a natural lordotic curve.

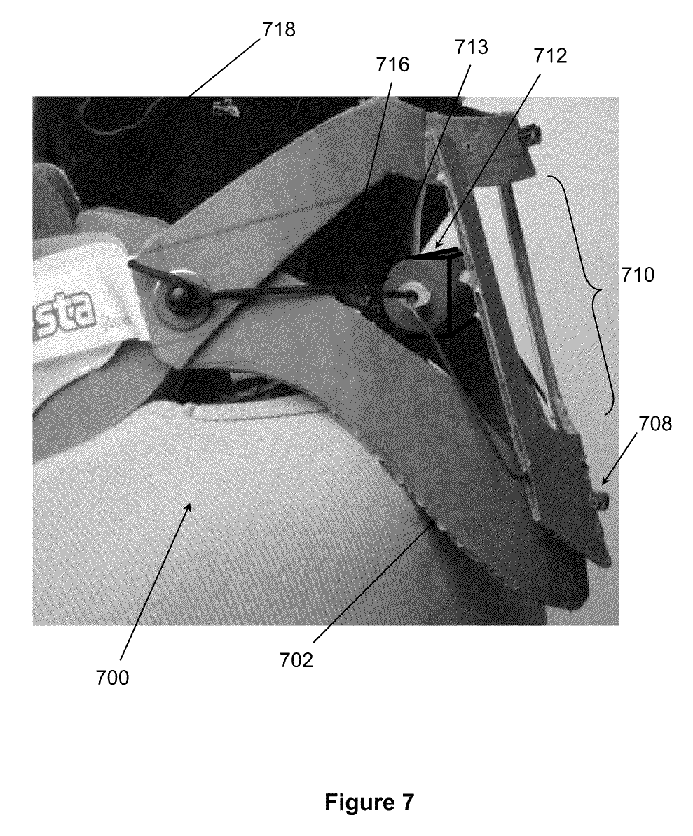

Alternatively as shown in FIG. 7, the member 112 could have a substantially rectangular cross-section with at least one rounded edge, have a cross section of other commercially suitable shapes, or lack a rounded edge altogether.

In some contemplated embodiments, member 112 can extend anteriorly through back portion 102. In other contemplated embodiments, the wearer can detach member 112 from the back portion 102 of collar 100.

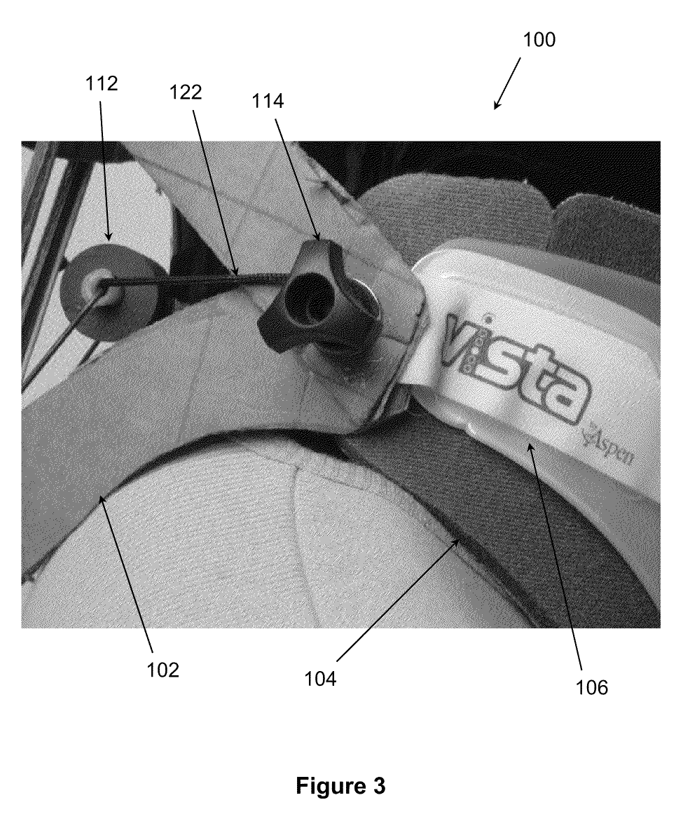

The collar 100 can include one or more mechanisms 114 shown in FIGS. 3-5 adapted to re-position a prominence of member 112 superiorly, inferiorly, anteriorly, and posteriorly relative to the back portion 102 while the collar 100 is installed on the wearer. For example, by rotating the mechanism 114, a cable 122 can be tightened or loosened which in turn increases or decreases the force of member 112 against the neck 116, respectively. This ability to manipulate the member 112 in a variety of directions is especially advantageous in that the direction and amount of pressure applied can be adjusted to suit each particular wearer's needs. Thus, a single cervical collar 100 could be adapted for differently sized and shaped wearers.

Although mechanism 114 is shown as a dial, it is contemplated that the mechanism 114 could be a strap or any other suitable mechanism for repositioning the prominence of member 112. In embodiments in which the mechanism 114 is a dial, the dial could be continuously variable or have discrete stops.

Optionally, collar 100 can include at least one sensor 120 configured to detect a pressure being applied by the member 112 to the neck 116. The sensor 120 can be disposed anywhere on the collar 100 such that the pressure applied to the neck 116 can be determined. Any commercially suitable pressure sensor could be used. In this instance, and where other upper limits are not expressly stated, the reader should infer a reasonable upper limit. In this instance, for example, a commercially reasonable upper limit is about ten.

FIG. 7 illustrates a collar 700 having a member 712 that has a substantially rectangular cross-section with rounded edge 713. With respect to the remaining numerals in FIG. 7, the same considerations for like components with like numerals of FIG. 1 apply.

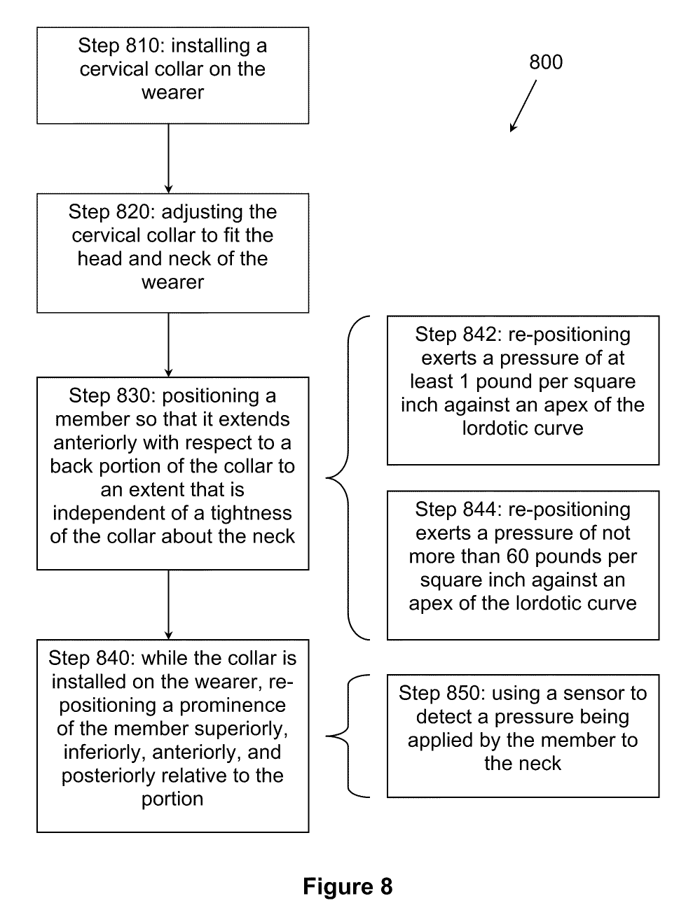

In FIG. 8, a method 800 for enforcing a lordotic curve in the cervical spine of a wearer can include the step 810 of installing a cervical collar on the wearer. In step 820, the collar can be adjusted to fit the wearer's head and neck.

In step 830, a member can be positioned so that the member extends anteriorly with respect to a back portion of the collar to an extent that is independent of a tightness of the collar about the neck. In step 840, while the collar is installed on the wearer, a prominence of the member can be repositioned superiorly, inferiorly, anteriorly, and posteriorly relative to the back portion. The prominence of the member can be advantageously re-positioned so as to exert a pressure of at least 1 lb/in² against an apex of the lordotic curve in step 842. Preferably, in step 844, the member can be repositioned to exert a pressure of not more than 60 lbs/in² against an apex of the lordotic curve.

Optionally in step 850, a sensor can be used to detect a pressure being applied by the member to the wearer's neck. This is beneficial, as the sensor can be used to alert a user if the collar is under- or over-tightened against the neck. The sensor can be disposed anywhere on the collar such that the pressure applied to the neck can be determined.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A cervical collar adjustable with respect to the head and neck of a wearer, comprising:
a back portion positionable posterior to the neck;
a member that extends anteriorly relative to the back portion to an extent that is independent of a tightness of the collar about the neck; and
a mechanism adapted to reposition a prominence of the member superiorly, inferiorly, anteriorly, and posteriorly relative to the portion while the collar is installed on the wearer.

2. The cervical collar of claim 1, further comprising a front portion coupled to the back portion with a tightenable strap.

3. The cervical collar of claim 1, further comprising a chest piece that supports at least a portion of the weight of the head.

4. The cervical collar of claim 1, further comprising a front portion that is continuous with the back portion.

5. The cervical collar of claim 1, further comprising a dial mechanism configured to adjust the tightness of the collar about the neck.

6. The cervical collar of claim 5, wherein the dial mechanism includes discrete stops.

7. The cervical collar of claim 1, wherein the member extends anteriorly through the back portion.

8. The cervical collar of claim 1, wherein the member is wearer-detachable from the back portion.

9. The cervical collar of claim 1, wherein the member has at least one substantially rectangular cross-section, with at least one rounded edge.

10. The cervical collar of claim 1, further comprising a sensor that detects a pressure being applied by the member to the neck.

11. A method of enforcing a lordotic curve in the cervical spine of a wearer, comprising:
installing a cervical collar on the wearer;
adjusting the cervical collar to fit the head and neck of the wearer;
positioning a member so that it extends anteriorly with respect to a back portion of the collar to an extent that is independent of a tightness of the collar about the neck; and
while the collar is installed on the wearer, repositioning a prominence of the member superiorly, inferiorly, anteriorly, and posteriorly relative to the back portion.

12. The cervical collar of claim 11, wherein the step of repositioning exerts a pressure of at least 1 pound per square inch against an apex of the lordotic curve.

13. The cervical collar of claim 11, wherein the step of repositioning exerts a pressure of not more than 60 pounds per square inch against an apex of the lordotic curve.

14. The cervical collar of claim 11, further comprising using a sensor to detect a pressure being applied by the member to the neck.
